(19) United States
(12) Patent Application Publication
(10) Pub. No.: US 2025/0228514 A1
(43) Pub. Date: Jul. 17, 2025

(54) MEDICAL IMAGE CAPTURING APPARATUS AND CONTROL METHOD OF THE SAME

(71) Applicant: FUJIFILM Corporation, Tokyo (JP)

(72) Inventor: Naoko TAKAGI, Tokyo (JP)

(73) Assignee: FUJIFILM Corporation, Tokyo (JP)

(21) Appl. No.: 18/981,690

(22) Filed: Dec. 16, 2024

(30) Foreign Application Priority Data
Jan. 16, 2024 (JP) 2024-004435

(51) Int. Cl. A61B 6/00; A61B 5/00; A61B 5/16; A61B 6/03; G06T 7/70

(52) U.S. Cl. CPC A61B 6/5223; A61B 5/0033; A61B 5/163; A61B 5/742; A61B 6/03; G06T 7/70; G06T 2207/10081

(57) ABSTRACT

A medical image capturing apparatus includes: a bed on which a subject is placed; a scan gantry section including a detection unit that detects a signal obtained from the subject; an image generation unit that generates a medical image using a detection signal transmitted from the detection unit; a display unit that displays the medical image; a camera that images the subject on the bed; an imaging range calculation unit that calculates an imaging range, which is a range in which the subject is imaged, based on a camera image obtained by the camera; a designated position acquisition unit that acquires a designated position which is a position designated by an action of an operator who is near the subject; and a cross-sectional image generation unit that generates a cross-sectional image to be displayed on a monitor viewed by the operator based on the imaging range and the designated position.

### CROSS-REFERENCE TO RELATED APPLICATION

The present application claims priority from Japanese Patent Application JP 2024-004435 filed on Jan. 16, 2024, the content of which is hereby incorporated by reference into this application.

### BACKGROUND

### Technical Field

The present invention relates to a medical image capturing apparatus that captures a medical image of a subject, and particularly relates to a technique for an operator who is near the subject to designate a position of a cross-sectional image.

### 2. DESCRIPTION OF THE RELATED ART

The medical image capturing apparatus is an apparatus that detects a signal obtained from a subject, for example, X-rays transmitted through the subject, nuclear magnetic resonance signals generated from the subject with a detection unit to capture a tomographic image, which is a medical image used for diagnosis or the like of the subject. For image diagnosis, it is preferable that a medical image is quickly displayed at a position desired by an operator.

JP1997-173352A (JP-H9-173352A) discloses that a coordinate system of a subject fixed by a fixing device and a coordinate system of a medical image captured by a medical image capturing apparatus are associated with each other using external shape information of the subject, and that a point designated in one coordinate system is displayed in the other coordinate system. For example, a point designated by a mouse pointer on the medical image is displayed on a body surface of the subject by light emitted from a light guide, or a point designated by a pointing device on the body surface or inside the body of the subject is indicated as a mark on the medical image.

However, in JP1997-173352A (JP-H9-173352A), consideration for the operator who is near the subject to designate the position of the cross-sectional image is insufficient. The operator who is near the subject can check a condition of the subject, but cannot operate the mouse pointer that moves by means of an operation unit. In addition, the pointing device that designates a point on the body surface or inside the body of the subject is not common.

Therefore, an object of the present invention is to provide a medical image capturing apparatus and a control method of the same that enable an operator who is near a subject to easily designate a position of a cross-sectional image to be displayed.

### SUMMARY

In order to achieve the above-described object, the present invention provides a medical image capturing apparatus comprising: a bed on which a subject is placed; a scan gantry section including a detection unit that detects a signal obtained from the subject; an image generation unit that generates a medical image using a detection signal transmitted from the detection unit; a display unit that displays the medical image; a camera that images the subject on the bed; an imaging range calculation unit that calculates an imaging range, which is a range in which the subject is imaged, based on a camera image obtained by the camera; a designated position acquisition unit that acquires a designated position which is a position designated by an action of an operator who is near the subject; and a cross-sectional image generation unit that generates a cross-sectional image to be displayed on a monitor viewed by the operator based on the imaging range and the designated position.

In addition, the present invention provides a control method of a medical image capturing apparatus including a bed on which a subject is placed, a scan gantry section including a detection unit that detects a signal obtained from the subject, an image generation unit that generates a medical image using a detection signal transmitted from the detection unit, a display unit that displays the medical image, and a camera that images the subject on the bed, the control method comprising: an imaging range calculation step of calculating an imaging range, which is a range in which the subject is imaged, based on a camera image obtained by the camera; a designated position acquisition step of acquiring a designated position which is a position designated by an action of an operator who is near the subject; and a cross-sectional image generation step of generating a cross-sectional image to be displayed on a monitor viewed by the operator based on the imaging range and the designated position.

According to the present invention, it is possible to provide a medical image capturing apparatus and a control method of the same that enable an operator who is near a subject to designate a position of a cross-sectional image to be displayed.

### BRIEF DESCRIPTION OF THE DRAWINGS

FIG. 1 is a diagram showing an example of an overall configuration of an X-ray CT apparatus of Example 1.

FIGS. 2A and 2B are diagrams illustrating a position of an operator and a bed.

FIG. 3 is a diagram showing an example of functional blocks of Example 1.

FIG. 4 is a diagram showing an example of a flow of processing of Example 1.

FIGS. 5A to 5D are diagrams supplementarily illustrating the example of the flow of the processing of Example 1.

FIG. 6 is a diagram illustrating a position of a line of sight of the operator.

FIG. 7 is a diagram illustrating a multi-sectional image generated from a plurality of tomographic images.

FIG. 8 is a diagram showing an example of a flow of processing of Example 2.

FIG. 9 is a diagram illustrating designation of a sagittal image.

FIG. 10 is a diagram illustrating designation of a coronal image.

### DESCRIPTION OF THE PREFERRED EMBODIMENTS

Hereinafter, examples of a medical image capturing apparatus according to an embodiment of the present invention will be described with reference to the accompanying drawings. The medical image capturing apparatus is an apparatus that detects a signal obtained from a subject, for example, X-rays transmitted through the subject, nuclear magnetic resonance signals generated from the subject to capture a medical image used for diagnosis or the like of the subject. Hereinafter, as an example of the medical image capturing apparatus, an X-ray computed tomography (CT) apparatus that captures a tomographic image of the subject by acquiring X-ray projection images of the subject at various projection angles will be described.

### Example 1

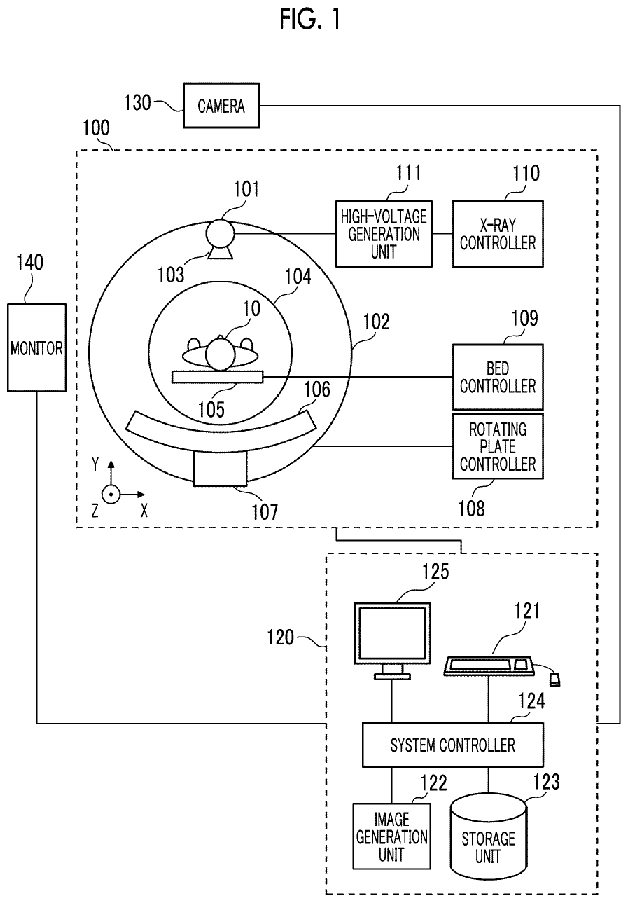

An overall configuration of an X-ray CT apparatus of Example 1 will be described with reference to FIG. 1. The X-ray CT apparatus comprises a scan gantry section 100, an operation unit 120, a camera 130, and a monitor 140. The scan gantry section 100, the camera 130, and the monitor 140 are installed in an imaging room surrounded by a shielding material that blocks X-rays, and the operation unit 120 is installed in an operation room located outside the imaging room. A body axis direction of a subject 10 is referred to as a Z axis, a horizontal direction orthogonal to the Z axis is referred to as an X axis, and a vertical direction orthogonal to the Z axis is referred to as a Y axis.

The scan gantry section 100 comprises an X-ray source 101, a rotating plate 102, a collimator 103, an X-ray detector 106, a data collection unit 107, a bed 105, a rotating plate controller 108, a bed controller 109, an X-ray controller 110, and a high-voltage generation unit 111. The X-ray source 101 is a device that irradiates the subject 10 placed on the bed 105 with X-rays and is, for example, an X-ray tube device. The collimator 103 is a device that restricts an irradiation range of the X-rays. The rotating plate 102 comprises an opening portion 104 through which the subject 10 placed on the bed 105 enters, and is also equipped with the X-ray source 101 and the X-ray detector 106 and rotates the X-ray source 101 and the X-ray detector 106 around the subject 10.

The X-ray detector 106 is a device that is disposed to face the X-ray source 101, that comprises a plurality of detection elements which detect X-rays transmitted through the subject 10, and that detects a spatial distribution of X-rays. In addition, the X-ray detector 106 functions as a detection unit that detects a signal obtained from the subject 10. The detection elements of the X-ray detector 106 are arranged two-dimensionally in a rotation direction and a rotation axis direction of the rotating plate 102. The data collection unit 107 is a device that collects the spatial distribution of X-rays detected by the X-ray detector 106 as digital data.

The rotating plate controller 108 is a device that controls rotation and tilt of the rotating plate 102. The bed controller 109 is a device that controls up, down, front, back, left, and right movements of the bed 105. The high-voltage generation unit 111 is a device that generates a high voltage applied to the X-ray source 101. The X-ray controller 110 is a device that controls an output of the high-voltage generation unit 111. The rotating plate controller 108, the bed controller 109, and the X-ray controller 110 are, for example, micro-processing units (MPUs) or the like.

The operation unit 120 comprises an input unit 121, an image generation unit 122, a display unit 125, a storage unit 123, and a system controller 124. The input unit 121 is a device that is used to input examination data such as a name of the subject 10, an examination date and time, and an imaging condition, and is, for example, a keyboard, a pointing device, or a touch panel. The image generation unit 122 is a device that generates a tomographic image using the digital data collected by the data collection unit 107 or generates a multi-sectional image using a plurality of tomographic images, and is, for example, an MPU or a graphics processing unit (GPU). The display unit 125 is a device that displays the tomographic image or the like generated by the image generation unit 122, and is, for example, a liquid crystal display or a touch panel. The storage unit 123 is a device that stores the digital data collected by the data collection unit 107, the tomographic image generated by the image generation unit 122, a program to be executed by the system controller 124, data to be used by the program, and the like, and is, for example, a hard disk drive (HDD) or a solid state drive (SSD). The system controller 124 is a device that controls each unit such as the rotating plate controller 108, the bed controller 109, and the X-ray controller 110, and is, for example, a central processing unit (CPU).

The high-voltage generation unit 111 generates a tube voltage, which is a high voltage applied to the X-ray source 101, based on the imaging condition set via the input unit 121, whereby X-rays corresponding to the imaging condition are emitted to the subject 10 from the X-ray source 101. The X-ray detector 106 detects the X-rays emitted from the X-ray source 101 and transmitted through the subject 10 with a large number of detection elements, and acquires a spatial distribution of the transmitted X-rays. The rotating plate 102 is controlled by the rotating plate controller 108 to rotate based on the imaging condition input through the input unit 121, particularly a rotation speed or the like. The bed 105 is controlled by the bed controller 109 and moves relative to the rotating plate 102 to move an imaging position designated with respect to the subject 10 to an imaging field of view, which is a range in which the transmitted X-rays are detected.

By repeating the irradiation of the X-rays from the X-ray source 101 and the detection of the X-rays using the X-ray detector 106 together with the rotation of the rotating plate 102, projection data, which is an X-ray projection image of the subject 10, is measured at various projection angles. The projection data is associated with a view representing each projection angle and a channel number and a column number, which are detection element numbers of the X-ray detector 106. The measured projection data is transmitted to the image generation unit 122. The image generation unit 122 performs back-projection processing on a plurality of projection data to generate a tomographic image. The generated tomographic image is displayed on the display unit 125 or stored in the storage unit 123 as a medical image. Which medical image out of the plurality of generated medical images is displayed on the display unit 125 is designated by operating the input unit 121.

The camera 130 is a device that images the subject 10 placed on the bed 105 together with the bed 105 from above, and is provided on a ceiling of the imaging room. The camera image captured by the camera 130 is used to calculate a position of the subject 10 with respect to the bed 105 or the scan gantry section 100 or to calculate a position of the operator who is near the subject 10. The camera image may be stored in the storage unit 123.

The monitor 140 is a device that displays a tomographic image or a multi-sectional image, and is provided, for example, on a side of the bed 105 so that the operator who is near the subject 10 can check the tomographic image or the like. The monitor 140 may be a gantry monitor provided in the scan gantry section 100 or a tablet terminal placed at an operator's hand.

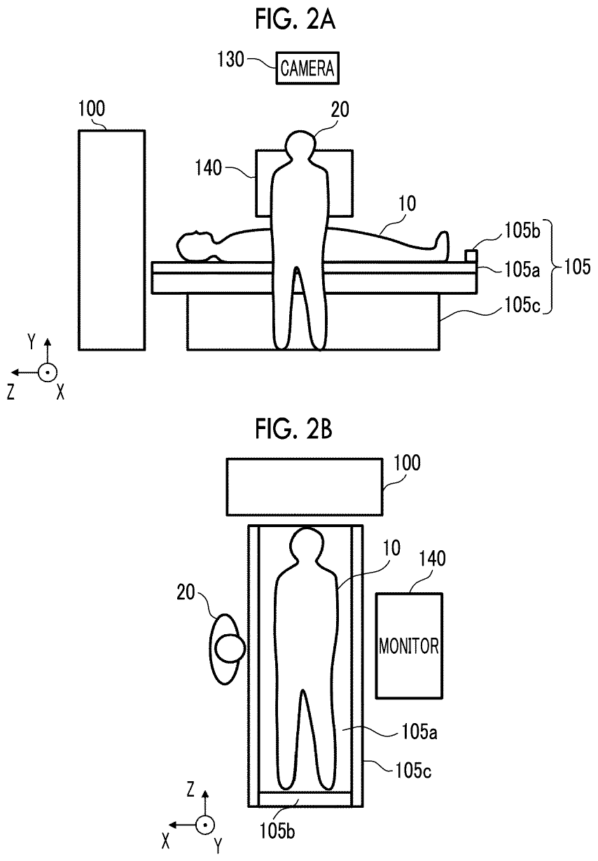

The position of the operator 20 and the bed 105 will be described with reference to FIGS. 2A and 2B. FIG. 2A is a view of the subject 10 placed on the bed 105 as viewed from the side, and FIG. 2B is a view of the subject 10 as viewed from above. The operator 20 is located near the subject 10 in a position where the operator 20 can see the monitor 140, in order to check a condition of the subject 10 or to perform a treatment on the subject 10.

The bed 105 has a top plate 105a, a grip 105b, and a base 105c. The top plate 105a is a plate on which the subject 10 is placed, and slides on the base 105c in the body axis direction of the subject 10. By sliding the top plate 105a, the imaging position of the subject 10 can be moved to the imaging field of view of the scan gantry section 100. The grip 105b is provided at an end part of the top plate 105a in the Z axis direction and is used in a case in which the top plate 105a is manually slid. The base 105c is a table installed on a floor surface of the imaging room, and moves up and down and also moves the top plate 105a left and right.

The operator 20 who is near the subject 10 can check the medical image of the subject 10 by viewing the monitor 140, but cannot operate the input unit 121 of the operation unit 120, so that the operator 20 cannot designate a medical image to be displayed on the monitor 140. Therefore, in Example 1, a configuration is adopted in which the operator 20 who is near the subject 10 can designate the medical image to be displayed on the monitor 140.

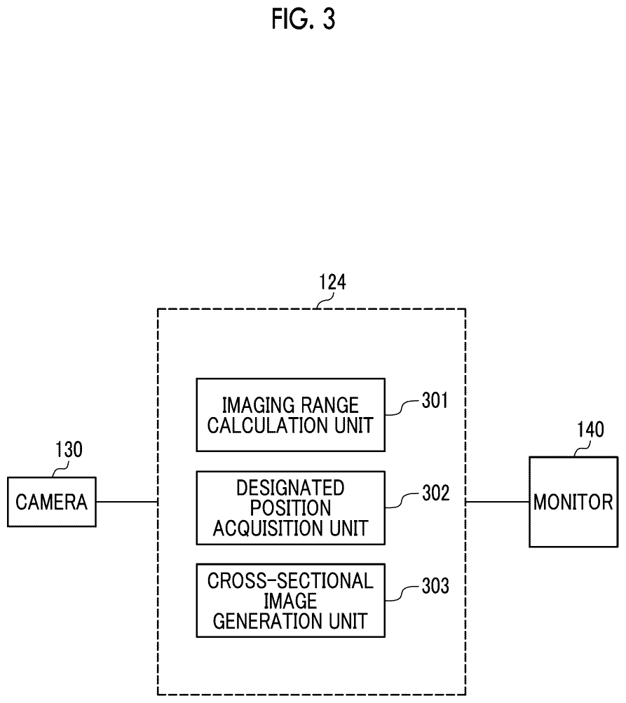

An example of functional blocks of Example 1 will be described with reference to FIG. 3. These functional blocks may be configured with dedicated hardware formed of an application specific integrated circuit (ASIC), a field-programmable gate array (FPGA), or the like, or may be configured with software that operates on the system controller 124. In the following description, a case in which the functional blocks of Example 1 are configured with software will be described.

In Example 1, an imaging range calculation unit 301, a designated position acquisition unit 302, and a cross-sectional image generation unit 303 are provided. Hereinafter, each unit will be described.

The imaging range calculation unit 301 calculates an imaging range, which is a range in which the subject 10 is imaged, based on the camera image obtained by the camera 130. The camera image is a frame image in a video and is a digitized image.

The designated position acquisition unit 302 acquires a designated position which is a position designated by an action of the operator 20 who is near the subject 10. The designated position may be obtained based on the camera image.

The cross-sectional image generation unit 303 generates a cross-sectional image to be displayed on the monitor 140 based on the imaging range calculated by the imaging range calculation unit 301 and the designated position acquired by the designated position acquisition unit 302.

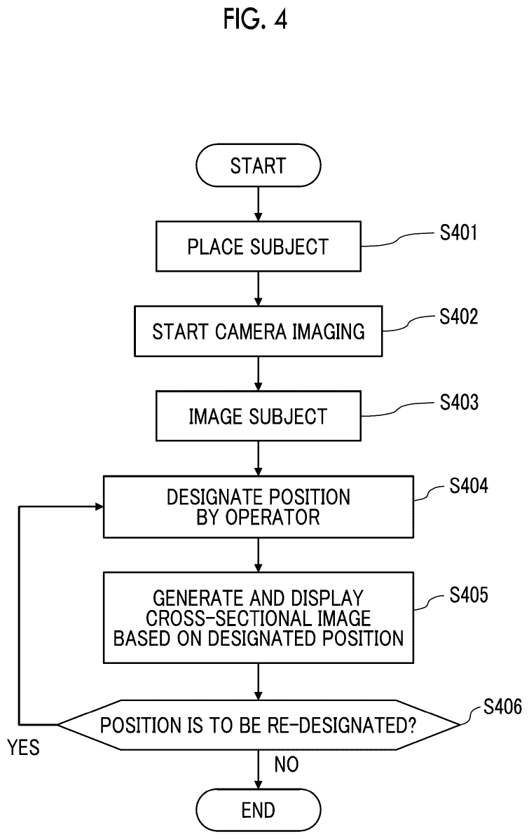

An example of a flow of processing of Example 1 will be described step by step with reference to FIG. 4.

### S401

The subject 10 is placed on the bed 105.

### S402

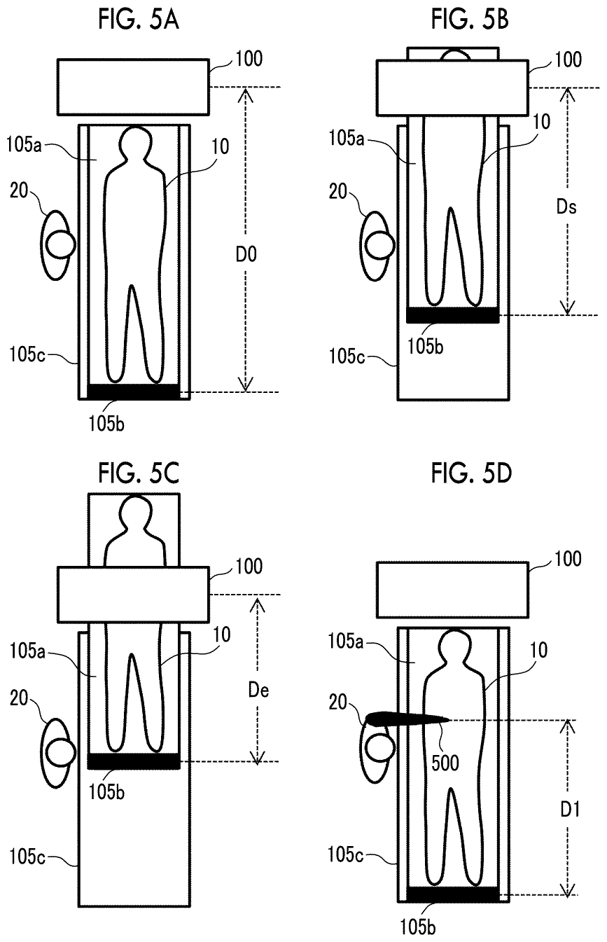

The camera 130 starts imaging of the subject 10. The camera 130 images the bed 105 and the scan gantry section 100 as well as the subject 10. The camera image captured by the camera 130 is transmitted to the system controller 124 and is used for calculating the position of the subject 10 with respect to the bed 105 or the scan gantry section 100. FIG. 5A illustrates a camera image used for calculating a distance DO between the grip 105b of the bed 105 and the scan gantry section 100. That is, the distance DO is a distance from the grip 105b to the center of the imaging field of view.

### S403

After the camera 130 starts the imaging in S402, a tomographic image of the subject 10 is captured. Even during the capturing of the tomographic image, the camera 130 continues to capture images. FIG. 5B illustrates a camera image in a case in which the subject 10 has reached an imaging start position. In addition, FIG. 5C illustrates a camera image in a case in which the subject 10 has reached an imaging end position.

The imaging range calculation unit 301 calculates the imaging range based on the camera images illustrated in FIGS. 5B and 5C. More specifically, from the camera image in FIG. 5B, the imaging range calculation unit 301 calculates a distance Ds between the grip 105b and the scan gantry section 100 in a case in which the subject 10 has reached the imaging start position. In addition, from the camera image in FIG. 5C, the imaging range calculation unit 301 calculates a distance De between the grip 105b and the scan gantry section 100 in a case in which the subject 10 has reached the imaging end position. Since the distance Ds corresponds to the imaging start position and the distance De corresponds to the imaging end position, the imaging range, which is a range from the imaging start position to the imaging end position, is represented by the distance from the grip 105b.

### S404

A position of the cross-sectional image to be displayed on the monitor 140 is designated by an action of the operator 20. For the designation of the position by the action, for example, a hand 500 of the operator 20 is used. By using the hand 500 to designate the position, it is possible to avoid the need to add a new device such as a pointing device. The hand 500 of the operator 20 is imaged by the camera 130 together with the bed 105. FIG. 5D illustrates a camera image in a case in which the operator 20 designates the position.

The designated position acquisition unit 302 acquires a designated position, which is a position designated by the operator 20, based on the camera image illustrated in FIG. 5D. More specifically, from the camera image in FIG. 5D, the designated position acquisition unit 302 acquires a distance D1 between the hand 500 of the operator 20 and the grip 105b. Since the distance D1 corresponds to the position of the hand 500 of the operator 20, the designated position is represented by the distance from the grip 105b. The position of the cross-sectional image to be displayed on the monitor 140 is not limited to be designated by the hand 500 of the operator 20.

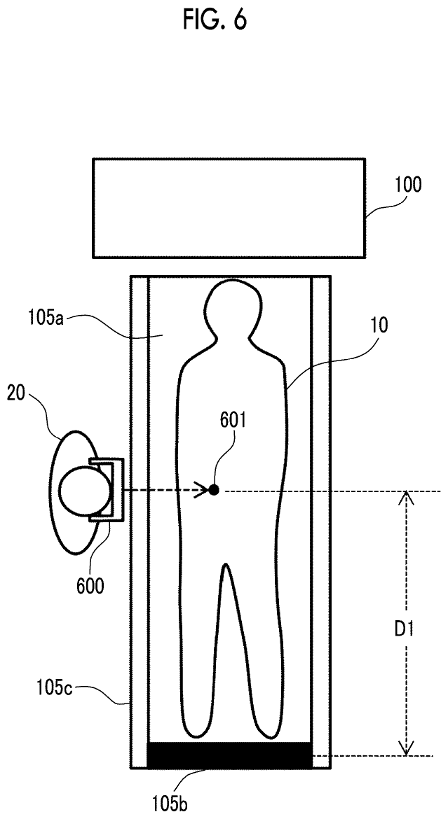

The acquisition of the designated position based on a line-of-sight position 601 of the operator 20 will be described with reference to FIG. 6. The line-of-sight position 601 of the operator 20 is detected by an eyeglasses device 600 worn by the operator 20. The eyeglasses device 600 detects the line-of-sight position 601 of the operator 20 and transmits data of the line-of-sight position 601 to the designated position acquisition unit 302. The designated position acquisition unit 302 calculates a distance D1 between the line-of-sight position 601 and the grip 105b based on the line-of-sight position 601 transmitted from the eyeglasses device 600 and the camera image. Since the distance D1 corresponds to the position of the line-of-sight position 601 of the operator 20, the designated position is represented by the distance from the grip 105b. By acquiring the designated position based on the line-of-sight position 601, the operator 20 can work without moving the hand 500.

### S405

The cross-sectional image generation unit 303 generates a cross-sectional image to be displayed on the monitor 140 based on the imaging range calculated in S403 and the designated position acquired in S404. For example, the cross-sectional image generation unit 303 selects a tomographic image closest to the designated position represented by the distance D1 from among a plurality of tomographic images captured in the imaging range represented by the distance Ds and the distance De, and uses the selected tomographic image as the cross-sectional image to be displayed on the monitor 140. Alternatively, two tomographic images close to the designated position represented by the distance D1 are selected from among the plurality of tomographic images captured in the imaging range represented by the distance Ds and the distance De, and the cross-sectional image at the designated position is generated by the cross-sectional image generation unit 303 by performing interpolation processing using the two tomographic images. The generated cross-sectional image is displayed on the monitor 140 and is checked by the operator 20.

### S406

The operator 20 determines whether or not to re-designate the position of the cross-sectional image by checking the cross-sectional image to be displayed on the monitor 140. In a case in which the position of the cross-sectional image is not re-designated, the flow of processing ends, and in a case in which the position is re-designated, the process returns to S404, and the operator 20 moves the position of the hand 500 or the line-of-sight position 601. In a case in which the operator 20 continues to move the position of the hand 500 or the line-of-sight position 601, the cross-sectional images are generated and displayed at any time in S405, so that a plurality of cross-sectional images are scrolled. The scrolling of the plurality of cross-sectional images is not limited to the movement of the position of the hand 500 or the line-of-sight position 601, and may be executed by a foot controller provided at feet of the operator 20 or a voice recognition device that recognizes a voice of the operator 20.

With the flow of the processing described with reference to FIG. 4, the operator 20 who is near the subject 10 can designate the position of the cross-sectional image to be displayed on the monitor 140 installed in the imaging room. In addition, after checking the cross-sectional image to be displayed on the monitor 140, the operator 20 can display a cross-sectional image at another position on the monitor 140.

### Example 2

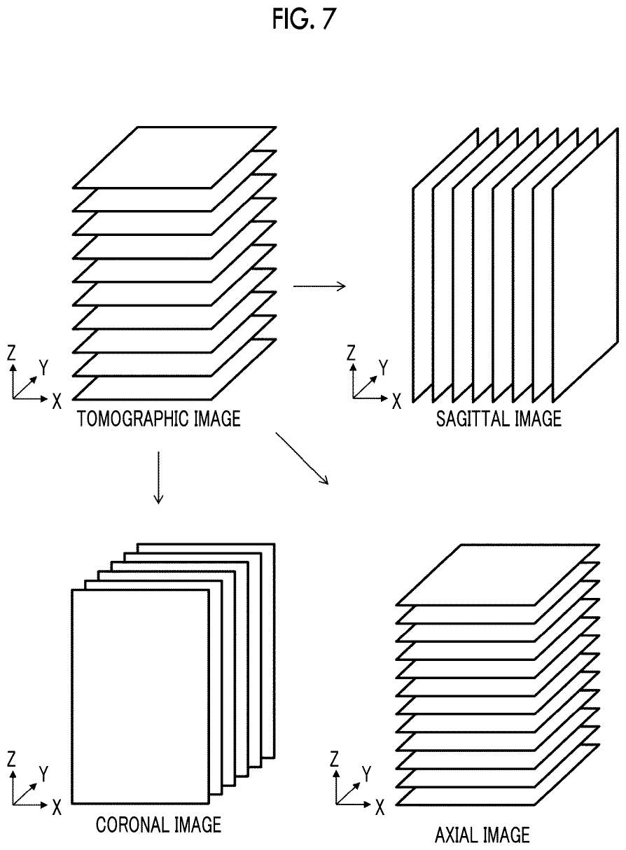

In Example 1, the generation of the cross-sectional image to be displayed on the monitor 140 based on the designated position, which is the position designated by the operator 20 who is near the subject 10, has been described. In a case in which a plurality of tomographic images are captured, a multi-sectional image can be generated using the plurality of tomographic images. That is, multi-sectional images such as a sagittal image parallel to a YZ plane, a coronal image parallel to a ZX plane, and an axial image parallel to an XY plane illustrated in FIG. 7 are generated from the plurality of tomographic images. In Example 2, a case will be described in which the orientation and position of the cross-sectional image to be displayed on the monitor 140 are designated by the action of the operator 20 who is near the subject 10. Since a part of the configurations or functions described in Example 1 can be applied to Example 2, the description of the same configurations and functions will be omitted.

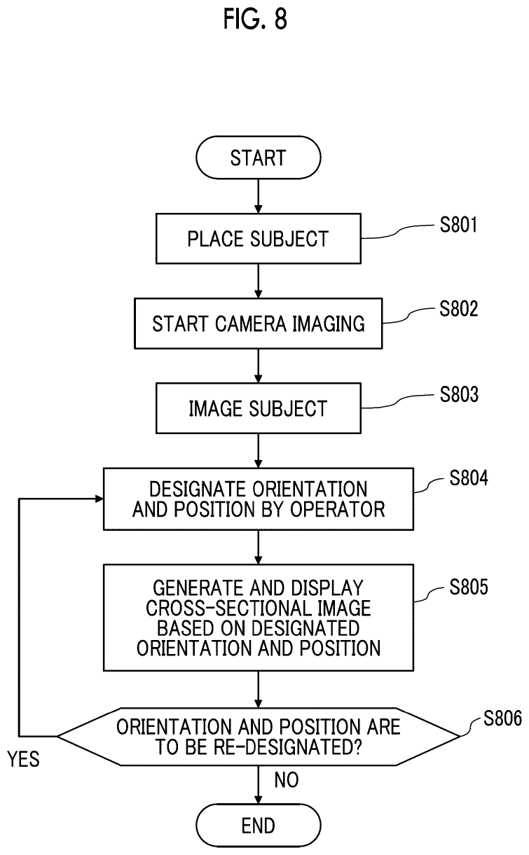

An example of a flow of processing of Example 2 will be described step by step with reference to FIG. 8. The description of the same process as in Example 1 will be simplified.

### S801 to S803

S801 to S803 are the same as S401 to S403 of Example 1.

### S804

The orientation and position of the cross-sectional image to be displayed on the monitor 140 are designated by an action of the operator 20. For the designation of the orientation and position by the action, for example, the hand 500 of the operator 20 is used. By using the hand 500 to designate the orientation and position, it is possible to avoid the need to add a new device such as a pointing device. The hand 500 of the operator 20 is imaged by the camera 130 together with the bed 105.

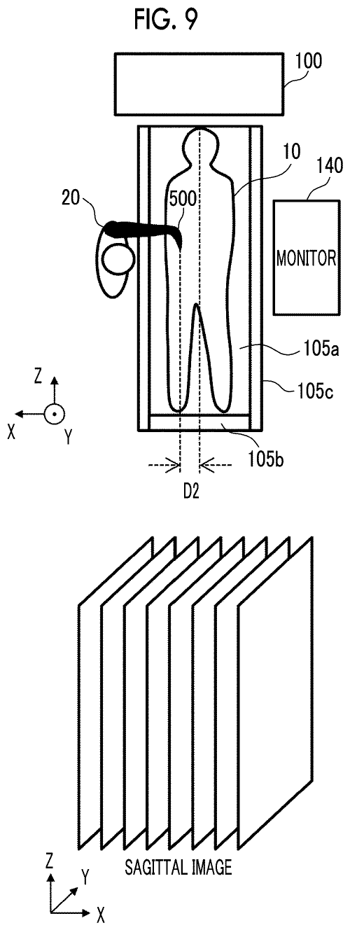

A case in which a sagittal image is designated will be described with reference to FIG. 9. The operator 20 designates a sagittal image by moving the hand 500, which is bent so that the palm is parallel to the YZ plane, above the subject 10, and designates the position of the sagittal image to be displayed on the monitor 140 by moving the hand 500 in the X axis direction. The YZ plane is a vertical plane parallel to the body axis direction of the subject 10. The position of the sagittal image is represented by, for example, a distance D2 between a center line extending in a longitudinal direction of the top plate 105a and the hand 500. The distance D2 is calculated by the designated position acquisition unit 302 based on the camera image.

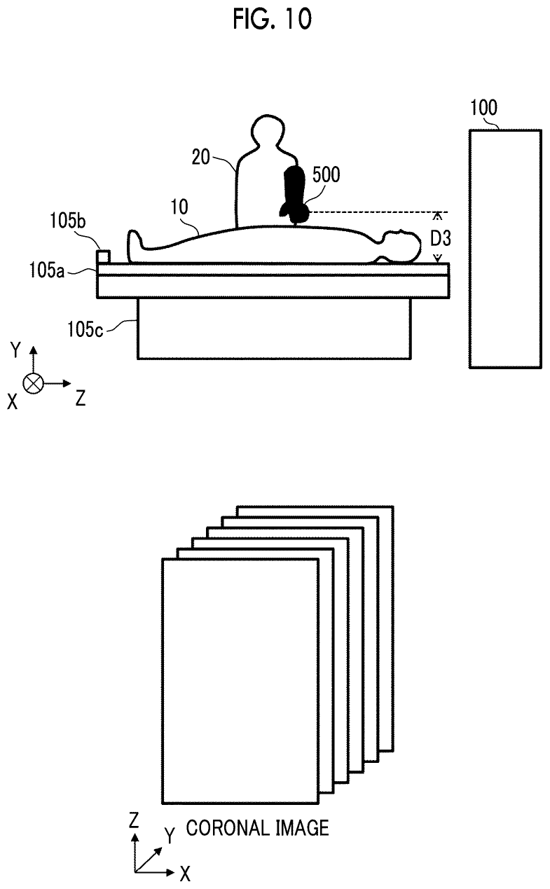

A case in which a coronal image is designated will be described with reference to FIG. 10. The operator 20 designates a coronal image by moving the hand 500 above the subject 10 with the palm parallel to the ZX plane, and designates the position of the coronal image to be displayed on the monitor 140 by moving the hand 500 in the Y axis direction. The ZX plane is a horizontal plane parallel to the body axis direction of the subject 10. The position of the coronal image is represented by, for example, a distance D3 between an upper surface of the top plate 105a and the hand 500. The distance D3 is calculated by the designated position acquisition unit 302 based on depth data of the camera image. A camera image captured by a camera installed on a side of the subject 10 may be used for the calculation of the distance D3.

The designation of the axial image is the same as in Example 1. That is, the operator 20 designates the axial image by moving the hand 500 above the subject 10 with the palm parallel to the XY plane, and designates the position of the axial image to be displayed on the monitor 140 by moving the hand 500 in the Z axis direction. The XY plane is a plane orthogonal to the body axis direction of the subject 10.

### S805

The cross-sectional image generation unit 303 generates a cross-sectional image to be displayed on the monitor 140 based on the imaging range calculated in S803 and the orientation and position designated in S804. The cross-sectional image generation unit 303 determines whether the palm is parallel to any of the XY plane, the YZ plane, or the ZX plane from the camera image, and generates any one of a sagittal image, a coronal image, or an axial image depending on a determination result. In a case in which the sagittal image is generated, the position of the cross-sectional image to be displayed on the monitor 140 is set based on the distance D2. Similarly, the position of the cross-sectional image to be displayed on the monitor 140 is set based on the distance D3 in a case of the coronal image and is set based on the distance D1 in a case of the axial image. The generated cross-sectional image is displayed on the monitor 140 and is checked by the operator 20.

### S806

The operator 20 determines whether or not to re-designate the orientation and position of the cross-sectional image by checking the cross-sectional image to be displayed on the monitor 140. In a case in which the orientation and position of the cross-sectional image are not re-designated, the flow of processing ends, and in a case in which the position is re-designated, the process returns to S804, and the operator 20 changes the orientation or the position of the hand 500.

With the flow of the processing described with reference to FIG. 8, the operator 20 who is near the subject 10 can designate the orientation and position of the cross-sectional image to be displayed on the monitor 140 installed in the imaging room. In addition, after checking the cross-sectional image to be displayed on the monitor 140, the operator 20 can display a cross-sectional image at a different orientation or position on the monitor 140.

The plurality of embodiments of the present invention have been described above. The present invention is not limited to the above-described embodiments, and the components can be modified and embodied without departing from the gist of the invention. Additionally, a plurality of components disclosed in the above-described embodiments may be combined as appropriate. Furthermore, some components may be deleted from all the components described in the above-described embodiments.

What is claimed is:

1. A medical image capturing apparatus comprising:
a bed on which a subject is placed;
a scan gantry section including a detection unit that detects a signal obtained from the subject;
an image generation unit that generates a medical image using a detection signal transmitted from the detection unit;
a display unit that displays the medical image;
a camera that images the subject on the bed;
an imaging range calculation unit that calculates an imaging range, which is a range in which the subject is imaged, based on a camera image obtained by the camera;
a designated position acquisition unit that acquires a designated position which is a position designated by a motion of an operator who is near the subject; and
a cross-sectional image generation unit that generates a cross-sectional image to be displayed on a monitor viewed by the operator based on the imaging range and the designated position.

2. The medical image capturing apparatus according to claim 1,
wherein the designated position acquisition unit acquires the designated position based on a position of an operator's hand obtained from the camera image.

3. The medical image capturing apparatus according to claim 2,
wherein the cross-sectional image generation unit generates the cross-sectional image based on an orientation of an operator's hand obtained from the camera image.

4. The medical image capturing apparatus according to claim 3,
wherein the cross-sectional image generation unit generates a sagittal image as the cross-sectional image in a case in which the orientation of the operator's hand is parallel to a vertical plane parallel to a body axis direction of the subject.

5. The medical image capturing apparatus according to claim 3,
wherein the cross-sectional image generation unit generates a coronal image as the cross-sectional image in a case in which the orientation of the operator's hand is parallel to a horizontal plane parallel to a body axis direction of the subject.

6. The medical image capturing apparatus according to claim 3,
wherein the cross-sectional image generation unit generates an axial image as the cross-sectional image in a case in which the orientation of the operator's hand is parallel to a plane orthogonal to a body axis direction of the subject.

7. The medical image capturing apparatus according to claim 1,
wherein the designated position acquisition unit acquires the designated position based on a line-of-sight position detected by an eyeglasses device worn by the operator.

8. A control method of a medical image capturing apparatus including a bed on which a subject is placed, a scan gantry section including a detection unit that detects a signal obtained from the subject, an image generation unit that generates a medical image using a detection signal transmitted from the detection unit, a display unit that displays the medical image, and a camera that images the subject on the bed, the control method comprising:
an imaging range calculation step of calculating an imaging range, which is a range in which the subject is imaged, based on a camera image obtained by the camera;
a designated position acquisition step of acquiring a designated position which is a position designated by an action of an operator who is near the subject; and
a cross-sectional image generation step of generating a cross-sectional image to be displayed on a monitor viewed by the operator based on the imaging range and the designated position.
